(19) United States
(12) Patent Application Publication
(10) Pub. No.: US 2021/0262045 A1
(43) Pub. Date: Aug. 26, 2021

(54) PRIMER PROBE COMBINATION, KIT, DETECTION METHOD AND APPLICATION FOR DETECTING SEPARATE CANDIDA TYPES

(71) Applicant: DYNAMIKER BIOTECHNOLOGY (TIANJIN) CO., LTD, TIANJIN (CN)

(72) Inventors: Zhixian WANG, TIANJIN (CN); Xiangyan YAN, TIANJIN (CN); Changzhong SHENG, TIANJIN (CN); Zeqi ZHOU, TIANJIN (CN); Yan SU, TIANJIN (CN)

(21) Appl. No.: 17/120,087

(22) Filed: Dec. 11, 2020

Related U.S. Application Data
(63) Continuation of application No. PCT/CN2019/112917, filed on Oct. 24, 2019.

(30) Foreign Application Priority Data
May 7, 2019 (CN) 201910375345.8

(51) Int. Cl. C12Q 1/6895; C12Q 1/686

(52) U.S. Cl. CPC C12Q 1/6895; C12Q 1/686

(57) ABSTRACT

The present invention provides a primer probe combination, a kit, a detection method and an application for detecting separate Candida types. The primer probe combination for detecting separate Candida types includes specific primers, detection probes and complementary probes for Candida albicans, Candida tropicalis, Candida parapsilosis, Candida krusei and Candida glabrata. The complementary probes and the detection probes are in incomplete complementary pairing. The primer probe combination in the present invention can detect and determine whether infection is caused by Candida in one step and simultaneously distinguish Candida albicans, Candida tropicalis, Candida parapsilosis, Candida krusei and Candida glabrata in the same fluorescence channel.

### CROSS-REFERENCE TO RELATED APPLICATIONS

This application is a continuation of International Patent Application No. PCT/CN2019/112917 with a filing date of Oct. 24, 2019, designating the United States, now pending, and further claims priority to Chinese Patent Application No. 201910375345.8 with a filing date of May 7, 2019. The content of the aforementioned applications, including any intervening amendments thereto, are incorporated herein by reference.

### INCORPORATION-BY-REFERENCE TO SEQUENCE LISTING

The Sequence listing in form of ASCII text filed via EFS-Web and hereby incorporated by reference into the specification in its entirety. The name of the text file containing the Sequence Listing is “SequenceListing”. The size of the text file is 11,018 bytes, and the text filed was created on Mar. 5, 2021.

### TECHNICAL FIELD

The present invention belongs to the technical field of biology, relates to a primer probe combination, a kit, a detection method and an application for detecting separate Candida types, and particularly relates to a primer probe combination, a kit, a detection method and an application for separately detecting Candida albicans, Candida tropicalis, Candida parapsilosis, Candida krusei and Candida glabrata.

### BACKGROUND OF THE PRESENT INVENTION

Candida is one of the most common conditioned pathogens in fungi, and mainly includes Candida albicans, Candida tropicalis, Candida glabrata, Candida parapsilosis and Candida krusei. The Candida accounts for over 80% of clinical medical samples. The Candida is often parasitic on human skin, oral cavity, vagina and intestinal mucosa. Candidiasis may be easily caused when immune functions of the body are reduced. Since a time period of distinguishing different Candida types by a culture method is long, Candida infection cannot be rapidly treated with clinically common broad-spectrum antifungal agents. Different Candida types have different antibiotic resistances. Different influences may be brought to the body if a unified treatment plan is adopted without distinguishing the types. Different strains are often detected by a polymerase chain reaction (PCR) in the prior art.

CN109055502A discloses a rapid multiple PCR identification and diagnosis detection method for invasive fungal infections based on cell free DNA (cfDNA). The method can identify invasive infections caused by clinically common and high-incidence Candida albicans, Candida tropicalis, Candida parapsilosis, Candida krusei, Candida glabrata and Aspergillus fumigtus. In the present invention, amplification primers are designed according to feature genome regions of various fungal genera and types; fluorescence detection probes that can distinguish the Candida types are designed according to amplified fragments; to-be-detected samples are subjected to real time PCR; and advantages such as high sensitivity of nested PCR and high specificity and multi-target property of multiple fluorescent hybridization probe PCR are integrated for identifying fungal strains. Although multiple pathogenic bacteria are identified by a one-step single-tube reaction in the method, multiple primers and probes exist in the reaction system, and analysis is performed by virtue of different fluorescence channels, thereby increasing detection cost and cycles.

CN101509037A discloses a rapid diagnostic kit for detecting four/more than four kinds of invasive Candida and an application method of the kit. The kit includes specific primers, a fluorescent quantitative PCR reaction solution of fluorescence probes corresponding to at least four kinds of Candida, and positive quality control and negative quality control of the at least four kinds of Candida; and the four kinds of Candida are judged according to Ct values detected by a fluorescent quantitative PCR detector. By virtue of dual control of high-specificity amplification of the primers and high specific hybridization of fluorescence probes, a false-positive rate is low. However, different fluorescence probes have different fluorophore modifications and detection channels, and thus detection cost and time are increased.

CN108034745A discloses a primer probe combination for detecting four kinds of Candida, and belongs to the technical field of microbiological detection. The primer probe combination includes a pair of primers and a probe. The pair of primers includes a forward primer and a reverse primer; and the probe is a sequence labeled with reporter fluorophores and quencher fluorophores. The pair of primers and the probe provided in the present invention can specifically and simultaneously detect four kinds of Candida such as Candida albicans, Candida tropicalis, Candida glabrata and Candida parapsilosis. However, specific strains corresponding to positive samples cannot be distinguished in the invention, i.e., types of the Candida cannot be distinguished.

Therefore, providing a method that is excellent in specificity, efficient, rapid, and high in sensitivity and can distinguish different Candida types has significances, and lays the foundation for accurate medication and epidemic disease study.

### SUMMARY OF PRESENT INVENTION

With respect to defects and actual requirements in the prior art, the present invention provides a primer probe combination, a kit, a detection method and an application for detecting separate Candida types. Whether infection is caused by Candida can be detected and determined at a time; and Candida albicans, Candida tropicalis, Candida parapsilosis, Candida krusei and Candida glabrata can be simultaneously distinguished in the same fluorescence channel.

To achieve the purpose, technical solutions of the present invention are as follows:

In a first aspect, the present invention provides a primer probe combination for separately detecting Candida. The primer probe combination includes specific primers, detection probes and complementary probes specified at Candida albicans, Candida tropicalis, Candida parapsilosis, Candida krusei and Candida glabrata respectively, wherein the complementary probes and the detection probes are in incomplete complementary pairing.

In the present invention, specific target sequences of common Candida albicans, Candida tropicalis, Candida parapsilosis, Candida krusei and Candida glabrata in invasive Candida are simultaneously amplified by multiple PCR, and analyzed and identified by a specially designed Taqman probe melting curve, thereby realizing multiple detections of the five kinds of common invasive Candida. The primer probe combination in the present invention is excellent in specificity and high in sensitivity and realizes separate detection of strains of the Candida.

In the present invention, the detection probes and the complementary probes are in incomplete complementary pairing. Non-complementary bases are not located in 5 base sequences at 5′ or 3′ ends of the primers, and non-complementary locations cannot be continuous. Tm values of the detection probe and the complementary probe in incomplete complementary pairing are calculated according to a Tm value calculation formula of the nearest neighbor method. The formula is as follows: Tm=ΔH*/(ΔS*+RlnC_{T}). In the formula, ΔH* and ΔS* are respectively standard enthalpy change and entropy change of a hybridization reaction; R is a gas constant of 1.987 cal /kmol; and C_{T} is a molar concentration of DNA molecules (when the DNA molecules are asymmetric sequences, the molar concentration is CT/4). A probe of the designed Tm values of the detection probe and the complementary probe is designed by utilizing the formula, and a series of complementary probe and detection probe combinations that can be clearly distinguished by the Tm values are further designed, thereby distinguishing different kinds of Candida by different Tm values in the same fluorescence channel.

Preferably, nucleotide sequences of the specific primers of the Candida albicans are shown as SEQ ID NO.1-2; nucleotide sequences of the specific primers of the Candida tropicalis are shown as SEQ ID NO.3-4; nucleotide sequences of the specific primers of the Candida parapsilosis are shown as SEQ ID NO.5-6; nucleotide sequences of the specific primers of the Candida krusei are shown as SEQ ID NO.7-8; and nucleotide sequences of the specific primers of the Candida glabrata are shown as SEQ ID NO.9-10.

Multiple pairs of primers and probes exist in the same reaction system; for detecting different target sequences, stability, reaction conditions and amplification efficiency among various sequences need to be balanced; with the increasing number of detected bacteria seeds, design difficulty of the primer probe combination is significantly increased, and the design is restricted by multiple conditions. In the present invention, by virtue of multiple sequence alignment specified at the Candida albicans, Candida tropicalis, Candida parapsilosis, Candida krusei and Candida glabrata, the specific primers are designed, so that the multiple primers can stably exist in the same reaction system, and the amplification efficiency is consistent, thereby increasing the detection specificity and sensitivity and avoiding cross impact among the primers.

Preferably, a nucleotide sequence of the detection probe of the Candida albicans is shown as SEQ ID NO.11; a nucleotide sequence of the detection probe of the Candida tropicalis is shown as SEQ ID NO.12; a nucleotide sequence of the detection probe of the Candida parapsilosis is shown as SEQ ID NO.13; a nucleotide sequence of the detection probe of the Candida krusei is shown as SEQ ID NO.14; and a nucleotide sequence of the detection probe of the Candida glabrata is shown as SEQ ID NO.15.

In the present invention, by aligning lots of nucleotide sequences on NCBI by virtue of ClustaX, the scope of aligned strains is as follows: 430 sequences of Candida albicans LC016565.1, HE860439.1, HE860438.1, LC201976.1, HG916992.1 and the like; 800 sequences of Candida parapsilosis AB109223.1, LT596112.1, LN864561.1, FM172980.1, LC390002.1 and the like; 350 sequences of Candida tropicalis KX664485.1, LT837798.1, JN542555.1, HM222942.1, FJ515173.1 and the like; 177 sequences of Candida krusei KU987874.1, KP674518.1, KX912139.1, FJ515204.1, EF568018.1 and the like; and 291 sequences of Candida glabrata HE993756.1, KU936094.1, KP780450.1, KM603625.1, LC011411.1, HG970737.1 and the like. By virtue of alignment of the 2048 sequences, highly conserved regions in the Candida types can be obtained to the full extent, thereby obtaining highly conserved regions and variable regions of different clinical isolates, further guiding primer design locations and increasing sensitivity of different isolates of different bacteria seeds. Selection of the target sequences is of great importance on the design of the primer probe combination. Consistent stability and amplification efficiency among different primers and probes and detection specificity and sensitivity need to be met simultaneously, and consistency of the reaction conditions should be further met. The more the types of the detected Candida are, the more the factors needing to be considered are. Meanwhile, the designed primer probe combination specified at detection of the five kinds of Candida is not equivalent to a primer probe combination specified at a single strain.

In the present invention, sequence regions such as 18S rRNA and 28S rRNA are preferred in regions of the specific target sequences of five kinds of to-be-detected Candida. Five Taqman probes and five complementary sequences thereof are respectively designed and prepared; and a pair of primer sequences is respectively designed at the periphery of the detection probe sequences, and the pair of primers includes a forward primer and a reverse primer. Specific targets are subjected to single or multiple single-color or multiple multicolor real-time fluorescent quantitative PCR amplification reactions with the designed primers.

Preferably, a nucleotide sequence of the complementary probe of the Candida albicans is shown as SEQ ID NO.16; a nucleotide sequence of the complementary probe of the Candida tropicalis is shown as SEQ ID NO.17; a nucleotide sequence of the complementary probe of the Candida parapsilosis is shown as SEQ ID NO.18; a nucleotide sequence of the complementary probe of the Candida krusei is shown as SEQ ID NO.19; and a nucleotide sequence of the complementary probe of the Candida glabrata is shown as SEQ ID NO.20.

In the present invention, a complementary probe is respectively designed specified at the detection probe of the five different kinds of Candida. The complementary probes and the detection probes are in incomplete complementary pairing and can form a double-stranded probe, so that the double-stranded probe specified at the different kinds of Candida has different melting properties; and the five different kinds of Candida can be distinguished by the characteristic melting temperature (Tm).

Preferably, the primer probe combination further includes an internal reference system.

Preferably, the internal reference system includes internal reference primers and an internal reference probe.

Preferably, nucleotide sequences of the internal reference primers are shown as SEQ ID NO.21-22.

Preferably, a nucleotide sequence of the internal reference probe is shown as SEQ ID NO.23.

Preferably, a quencher is modified at the 3′ end of the detection probe of the Candida albicans, Candida tropicalis, Candida parapsilosis, Candida krusei and Candida glabrata; and a fluorophore is modified at the 5′ end.

Preferably, the fluorophore includes any one or a combination of at least two of ALEX-350, Alexa Fluor 488, CY3, FAM, VIC, TET, CALGold540, JOE, HEX, CALFluorOrange560, TAMRA, CALFluorRed590, ROX, CALFluorRed610, TexasRed, CALFluorRed635, Quasar670, CYS, CY5.5, LC RED640 or Quasar705.

In the present invention, the fluorophores are modified at the 5′ ends of the detection probes specified at the different kinds of Candida and may be the same fluorophore or different fluorophores. When the same fluorophore is modified, the primer probe combination designed in the present invention can perform detection and analysis in the same fluorescence detection channel; according to different melting temperatures of the double-stranded probe formed by the detection probe and the complementary probe, melting curve analysis is conducted after the real-time fluorescent quantitative PCR amplification reaction; whether one or more of five kinds of pathogenic Candida exist in the to-be-detected samples is judged according to melting point changes of the Taqman probe; and the Candida bacteria seeds in the to-be-detected samples are identified by a Tm value-bacteria seed standard comparison table, i.e., the multiple single-color real-time fluorescent quantitative PCR reaction is carried out. When the different fluorophores are modified, the multiple multicolor real-time fluorescent quantitative PCR reaction can be carried out. In addition, a certain one of the five kinds of Candida can be detected alone, i.e., the single single-color real-time fluorescent quantitative PCR reaction can be carried out.

Preferably, the quencher modified at the 3′ end of the detection probe includes any one or a combination of at least two of TAMRA, DABCYL, BHQ-1, BHQ-2, BHQ-3 or Eclipse.

With respect to the different kinds of Candida, the used fluorophores and quenchers may be the same or different, but different fluorophores need different fluorescence detection channels. Moreover, spectral ranges of excited light and absorbed light of the different selected fluorophores should avoid interference. Preferably, the fluorophores and quenchers modified by the probe for detecting different kinds of the Candida are in the same group.

A combination of the fluorophore and the quencher may be typically and non-finitely the fluorophore FAM and the quencher TAMRA, the fluorophore HEX and the quencher BHQ-1, the fluorophore ROX and the quencher BHQ-2, and the fluorophore CY5 and the quencher BHQ-3, preferably the fluorophore FAM and the quencher TAMRA.

Preferably, the 3′ end of the complementary probe is modified with the quencher.

Preferably, the quencher modified at the 3′ end of the complementary probe includes any one or a combination of at least two of TAMRA, DABCYL, BHQ-1, BHQ-2, BHQ-3 or Eclipse.

Preferably, the complementary probe and the detection probe have the same quencher. The quencher of the probe may be different from the quencher of the complementary sequences, but spectral bands of the fluorophores need to be matched.

In the present invention, the quencher can be selectively modified at the 3′ end of the complementary probe. The detection probe is complementary to the target sequence. During design, the detection probe is labeled with the fluorophore at the 5′ end and labeled with the quencher at the 3′ end. The 3′ end of the complementary probe may be designed to include the quencher, may also be designed without the quencher. When the 3′ end of the complementary probe is not provided with the quencher, a fluorescence value is decreased along with unwinding of strands of the detection probe and the complementary probe during temperature rise. The reason of such a phenomenon is as follows: a linear single-stranded double-labeled oligonucleotide in the solution is of random coil: two ends of the oligonucleotide are close to each other occasionally, and thus energy of the fluorophore is absorbed by the quencher. However, when the detection probe is combined with the complementary probe, two ends of the probe are forced to separate by a labeled heterozygote, and interaction between labels at the two terminals is destructed, thereby changing the fluorescence value. If the complementary probe is labeled with the quencher at the 3′ end, the fluorescence value is decreased by hybridization between the probes. Moreover, when the temperature rises to cause double-stranded denaturation, the fluorescence value is increased, which is because the quencher and the fluorophore are closer to each other due to formation of the probe heterozygote and the fluorescence signal is effectively quenched.

In a second aspect, the present invention provides a kit for detecting separate Candida types. The kit includes the primer probe combination in the first aspect.

Preferably, the kit further includes negative quality control, positive quality control and auxiliary reagents.

Preferably, the negative quality control is plasmids containing arabidopsis DNA fragments; and a nucleotide sequence of the arabidopsis DNA fragments is shown as SEQ ID NO.24.

Preferably, the positive quality control is respectively a plasmid containing Candida albicans DNA fragments, a plasmid containing Candida tropicalis DNA fragments, a plasmid containing Candida parapsilosis DNA fragments, a plasmid containing Candida krusei DNA fragments, and a plasmid containing Candida glabrata DNA fragments.

In the present invention, both the negative quality control and the positive quality control adopt a common plasmid pMD19 in the field. It should be indicated that, any vector meeting plasmid construction of the negative quality control and the positive quality control may be used for replacing the pMD19. A special limitation is not given herein.

Preferably, nucleotide sequences of the Candida albicans DNA fragments, the Candida tropicalis DNA fragments, the Candida parapsilosis DNA fragments, the Candida krusei DNA fragments and the Candida glabrata DNA fragments are respectively shown as SEQ ID NO.25, SEQ ID NO.26, SEQ ID NO.27, SEQ ID NO.28 and SEQ ID NO.29.

Preferably, the auxiliary reagents include a Taq enzyme, dNTP, MgCl₂, DMSO and a buffer solution.

Preferably, the buffer solution includes Tris-HCl and KCl.

Preferably, a concentration of the Tris-HCl in the buffer solution is 15-20 mM, e.g., 15 mM, 16 mM, 17 mM, 18 mM, 19 mM or 20 mM.

Preferably, a concentration of the KCl in the buffer solution is 100-200 mM, e.g., 100 mM, 110 mM, 120 mM, 130 mM, 140 mM, 150 mM, 160 mM, 170 mM, 180 mM, 190 mM or 200 mM.

In a third aspect, the present invention provides a method for detecting separate Candida types by utilizing the kit in the second aspect. The method includes the following steps:

(1) extracting sample DNA;

(2) adding a Taq enzyme, dNTP, MgCl₂, DMSO, a buffer solution and the primer probe combination in the first aspect into the sample DNA in the step (1); and

(3) carrying out a real-time fluorescent PCR reaction.

Preferably, a final concentration of the DNA sample in the step (2) is 0.1 pg-10 ng, e.g., 0.1 pg, 0.5 pg, 1 pg, 2 pg, 3 pg, 4 pg, 5 pg, 10 pg, 20 pg, 30 pg, 40 pg, 50 pg, 60 pg, 70 pg, 80 pg, 90 pg, 100 pg, 200 pg, 300 pg, 400 pg, 500 pg, 600 pg, 700 pg, 800 pg, 900 pg, 1 ng, 2 ng, 3 ng, 4 ng, 5 ng, 6 ng, 7 ng, 8 ng, 9 ng or 10 ng.

Preferably, a final concentration of the Taq enzyme in the step (2) is 0.5-5 U, e.g., 0.5 U, 0.8 U, 1 U, 1.2 U, 1.5 U, 1.8 U, 2 U, 2.5 U, 3 U, 3.5 U, 4 U, 4.5 U or 5 U.

Preferably, a final concentration of the dNTP in the step (2) is 0.1-5 M, e.g., 0.1 M, 0.3 M, 0.5 M, 0.8 M, 1 M, 1.2 M, 1.5 M, 1.8 M, 2 M, 2.5 M, 2.8 M, 3 M, 3.5 M, 4 M, 4.5 M, 4.8 M or 5 M.

Preferably, a volume percentage of the DMSO in the step (2) is 1-8%, e.g., 1%, 2%, 3%, 4%, 5%, 6%, 7% or 8%, preferably 5%.

Preferably, a final concentration of specific primers of the five kinds of Candida in the step (2) is 50-500 nM, e.g., 50 nM, 60 nM, 70 nM, 80 nM, 90 nM, 100 nM, 120 nM, 150 nM, 180 nM, 200 nM, 250 nM, 300 nM, 350 nM, 400 nM, 450 nM or 500 nM.

Preferably, a final concentration of a detection probe of the five kinds of Candida in the step (2) is 50-500 nM, e.g., 50 nM, 60 nM, 70 nM, 80 nM, 90 nM, 100 nM, 120 nM, 150 nM, 180 nM, 200 nM, 250 nM, 300 nM, 350 nM, 400 nM, 450 nM or 500 nM.

Preferably, a final concentration of a complementary probe of the five kinds of Candida in the step (2) is 50-500 nM, e.g., 50 nM, 60 nM, 70 nM, 80 nM, 90 nM, 100 nM, 120 nM, 150 nM, 180 nM, 200 nM, 250 nM, 300 nM, 350 nM, 400 nM, 450 nM or 500 nM.

In the present invention, the specific primers specified at each kind of Candida in the PCR reaction system have the consistent final concentration; according to a combination ratio 1:1:1:1:1 of the five pairs of primers, the final concentration range is 50-500 nM; and the primer design in the present invention is designed to meet amplification efficiency consistency.

In the present invention, the detection probes specified at each kind of Candida in the PCR reaction system have the consistent final concentration; and according to a combination ratio 1:1:1:1:1 of the five probes, the final concentration range is 50-500 nM.

In the present invention, the complementary probes specified at each kind of Candida in the PCR reaction system have the consistent final concentration; and according to a combination ratio 1:1:1:1:1 of the five probes, the final concentration range is 50-500 nM.

Preferably, conditions of the real-time fluorescent PCR reaction in the step (3) are as follows:

(1′) pre-degeneration at 90-98° C. for 1-10 min, and 1 cycle;

(2′) degeneration at 90-98° C. for 10 s; extension at 55-60° C. for 35-45 s; and 40-50 cycles; and

(3′) heating at 35-97° C.; and 1 cycle.

Preferably, before the step (1′), the reaction further includes a preheating process: 50° C. for 2 min; and 1 cycle.

Preferably, the real-time fluorescent PCR reaction in the step (3) specifically includes the following steps:

(1″) preheating at 50° C. for 2 min, and conducting 1 cycle;

(2″) performing pre-degeneration at 95° C. for 10 min, and conducting 1 cycle;

(3″) performing degeneration at 95° C. for 10 s; performing extension at 58° C. for 40 s; acquiring a fluorescence signal during extension; and conducting 45 cycles;

(4″) maintaining the temperature at 95° C. for 5 s and at 35° C. for 1 min; raising the temperature to 97° C. at 0.2° C. per second; continuously acquiring the fluorescence signal for 5 times per ° C.

Preferably, after the step (3), the method further includes a judgment step; and judgment standards are as follows:

(a) if the Ct value of the detection channel is not greater than 35, the result is positive; and if the Ct value of the detection channel is greater than 35, the result is negative; and

(b) the positive result is further analyzed; by virtue of melting curve analysis, corresponding annealing temperature values Tm of the product and five kinds of positive quality control are contrasted; and if the Tm values are the same, to-be-detected samples are judged to contain Candida types corresponding to the positive quality control.

In a fourth aspect, the present invention provides an application of the primer probe combination in the first aspect and/or the kit in the second aspect in detection of separate Candida types or preparation of a reagent for detection of separate Candida types.

Compared with the prior art, the present invention has beneficial effects as follows:

(1) The primer probe combination in the present invention has high specificity and sensitivity, and can simultaneously detect the Candida albicans, Candida tropicalis, Candida parapsilosis, Candida krusei and Candida glabrata and distinguish specific bacteria seeds.

(2) The primers designed for the Candida albicans, Candida tropicalis, Candida parapsilosis, Candida krusei and Candida glabrata in the present invention meet the amplification efficiency consistency while meeting ordinary amplification requirements, so that the detection system is more stable and excellent in detection specificity, thereby avoiding mutual affects among the bacteria seeds due to amplification efficiency differences.

(3) The detection method in the present invention is high in sensitivity, can rapidly detect the Candida albicans, Candida tropicalis, Candida parapsilosis, Candida krusei and Candida glabrata in combination with the specific primer probe combination, can identify and distinguish different Candida types in the same fluorescence detection channel simultaneously, and can also distinguish the specific target sequences of the multiple Candida types in the same fluorescence channel in accordance with the melting point change of the Taqman probe.

(4) Melting curve analysis of the oligonucleotide probe may be directly conducted after the real-time fluorescent PCR amplification reaction, without an open-tube operation, and may also be conducted in a real-time fluorescent PCR instrument after ordinary PCR amplification.

(5) The melting curve analysis of the oligonucleotide probe belongs to non-expendable testing; and after the analysis is ended, the samples are maintained in a state after the PCR and can be repeatedly analyzed.

### DESCRIPTION OF THE DRAWINGS

FIG. 1 is a melting curve of a product detected by taking plasmids containing DNA fragments of Candida albicans, Candida tropicalis, Candida parapsilosis, Candida krusei and Candida glabrata as a template respectively;

FIG. 2 is an amplification curve corresponding to a standard plasmid of different concentrations containing DNA fragments of Candida albicans in Embodiment 4;

FIG. 3 is a melting curve corresponding to a standard plasmid of different concentrations containing DNA fragments of Candida albicans in Embodiment 4;

FIG. 4 is an amplification curve chart of different concentration gradients of Candida albicans in Embodiment 5; and

FIG. 5 is a linear relation diagram between different concentrations of Candida albicans and a Ct value in Embodiment 5.

### DETAILED DESCRIPTION OF PREFERRED EMBODIMENTS

To further describe technological means taken in the present invention and effects of the technological means, technical solutions in the present invention will be further described below through specific embodiments. However, the present invention is not limited in the scope of the embodiments.

Embodiment 1 Assembly of Kit

Nucleotide sequences of specific primers, detection probes and complementary probes for Candida albicans, Candida tropicalis, Candida parapsilosis, Candida krusei and Candida glabrata are shown in Table 1.

TABLE 1

| | SEQ | |
|---|---|---|
| Primer name | ID NO. | Nucleotide sequence 5′-3′ |
| Candida albicans forward | 1 | TGTTTGAGCGTCGTTTCT |
| primer | | |
| Candida albicans reverse | 2 | AAGTTCAGCGGGTAGTCC |
| primer | | |
| Candida tropicalis forward | 3 | GCCTGTTTGAGCGTCGTTTC |
| primer | | |
| Candida tropicalis reverse | 4 | CGCCTTACCACTACCGTCTT |
| primer | | |
| Candida parapsdosis forward | 5 | CTGTTTGAGCGTCGTTTCT |
| primer | | |
| Candida parapsdosis reverse | 6 | TAAGTTCAGCGGGTAGTCC |
| primer | | |
| Candida krusei forward primer | 7 | AGTACTACACTGCGTGAGCG |
| Candida krusei reverse primer | 8 | TGCGAGAACCAAGAGATCCG |
| Candida glabrata forward | 9 | AACGCAGCGAAATGCGATAC |
| primer | | |
| Candida glabrata reverse | 10 | GGAATACCAGAGGGCGCAAT |
| primer | | |
| Candida albicans detection | 11 | ATCACTGTACTTGTTCGCTATCGGT |
| probe | | |
| Candida tropicalis detection | 12 | TTTCCACTCATTGGTACAAACTCCA |
| probe | | |
| Candida parapsdosis detection | 13 | ACGCACACTCCCAGGTCTTT |
| probe | | |
| Candida krusei detection probe | 14 | TCCTCCGTCTATGCTCCGTTACCTGTT |
| Candida glabrata detection | 15 | AAACGCTTATTTTGCTAGTGGCCA |
| probe | | |
| Candida albicans | 16 | TAGTGACATGAACAAGCGATAACCA |
| complementary probe | | |
| Candida tropicalis | 17 | AAAGGCGAGAAACCAAGTTAGAGGT |
| complementary probe | | |
| Candida parapsdosis | 18 | TGCGAGTGAGGGACCAGAAA |
| complementary probe | | |
| Candida krusei complementary | 19 | AGGATGCACATACGAGACAATGG |
| probe | | ACAA |
| Candida glabrata | 20 | TTTGCGAATAAAACCATCACCGGT |
| complementary probe | | |
| Forward primer of internal | 21 | CTGTGAATGCCATTTCTC |
| reference primer | | |
| Reverse primer of internal | 22 | ACACCTCCTTTGGAATAG |
| reference primer | | |
| Internal reference probe | 23 | CCTAACCTACCCGCCTTGGATATT |

Negative quality control: the negative quality control is a plasmid containing arabidopsis DNA fragments; a nucleotide sequence of the arabidopsis DNA fragments is shown as SEQ ID NO.24; and the specific sequence is as follows:

CATGATTCAGCCAACATAACCCGACCCGTACAATTCTATTACGCGT
CTCCGGCGACTGACTATACTTGCCGTTCGGAGTTAGGGTTTTACTCCGAG
AGAAAATTGAGTCAGCGATGAATCCGTTGACAAGGTGAAGAAGACGCAG
AGCATTAACGCGAAAGAATTAGATCTAGGGATTTACGACGAAGCATCTTG
GCACGCCAAGTACAAAGATTCAGCCTACGTTTACGTCGGAGAATTACCTT
ACGATCTCACGGAGGGTGACCTCCTCGCCGTTTTCTCACAATATGGTGAA
GTTGTTGATTTGAATCTTGTTCGAGATAAAGGAACTGGGAGATCAAAAAG
ATTTGCGTTTGTTGCTTATGAAGATCAGAGAAGTACTAATCTTGCTGTTG
GATAATAAAAGTGGAGCATTGTGGTAAATACTTAAAGAGAGAAGAGGAAG
ATGAAGAGACGAAGCAGAAGAAGAGAGAAGCTCCGTGGTGTTTGCAGAG
CTTTTCAGAGAAAGGAGTGTACTCGTGGAGATTCTTGCAAATTTTCTCAC
GATGAAAATAGAGCTGCCAATACCGGGTGGGGTCACGAAGATCGTAGAA
GTTCCAAGTGAGAGATCAAGTAAGTAAATACCATAATGATGTAGAGGAT
AAATAGGGATTGTTCATATTGATATCCAAGTGGTAATGCTCTACATTGAA
GAATGGTTTCTAAGTTGTAGCTAGAATCTAATGGAAAATGTGATTTATTG
ACCATTAGCTTACACACCGGTGTTTTGCTCTGTATATGTCGATCTTATTT
TCAGTGTTCACCTTTAC.

Positive quality control: a plasmid containing Candida albicans DNA fragments, a plasmid containing Candida tropicalis DNA fragments, a plasmid containing Candida parapsilosis DNA fragments, a plasmid containing Candida krusei DNA fragments, and a plasmid containing Candida glabrata DNA fragments.

A nucleotide sequence of the Candida albicans DNA fragments is shown as SEQ ID NO.25, and is specifically as follows:

ATATCAATAAGCGGAGGAAAAGAAACCAACAGGGATTGCCTCAGT
AGCGGCGAGTGAAGCGGCAAAAGCTCAAATTTGAAATCTGGCGTCTTTG
GCGTCCGAGTTGTAATTTGAAGAAGGTATCTTTGGGCCCGGCTCTTGTCT
ATGTTCCTTGGAACAGGACGTCACAGAGGGTGAGAATCCCGTGCGATGA
GATGACCCGGGTCTGTGTAAAGTTCCTTCGACGAGTCGAGTTGTTTGGGA
ATGCAGCTCTAAGTGGGTGGTAAATTCCATCTAAAGCTAAATATTGGCGA
GAGACCGATAGCGAACAAGTACAGTGATGGAAAGATGAAAAGAACTTTG
AAAAGAGAGTGAAAAAGTACGTGAAATTGTTGAAAGGGAAGGGCTTGAGA
TCAGACTTGGTATTTTGCATGCTGCTCTCTCGGGGGCGGCCGCTGCGGTT
TACCGGGCCAGCATCGGTTTGGAGCGGCAGGATAATGGCGGAGGAATGT
GGCACGGCTTCTGCTGTGTGTTATAGCCTCTGACGATACTGCCAGCCTAG
ACCGAGGACTGCGGTTTTTACCTAGGATGTTGGCATAATGATCTTAAGTC
GC.

A nucleotide sequence of the Candida tropicalis DNA fragments is shown as SEQ ID NO.26, and is specifically as follows:

AAAAGAAACCAACAGGGATTGCCTTAGTAGCGGCGAGTGAAGCG
GCAAAAGCTCAAATTTGAAATCTGGCTCTTTCAGAGTCCGAGTTGTAATT
TGAAGAAGGTATCTTTGGGTCTGGCTCTTGTCTATGTTTCTTGGAACAGA
ACGTCACAGAGGGTGAGAATCCCGTGCGATGAGATGATCCAGGCCTATGT
AAAGTTCCTTCGAAGAGTCGAGTTGTTTGGGAATGCAGCTCTAAGTGGGT
GGTAAATTCCATCTAAAGCTAAATATTGGCGAGAGACCGATAGCGAACA
AGTACAGTGATGGAAAGATGAAAAGAACTTTGAAAAGAGAGTGAAAAA
GTACGTGAAATTGTTGAAAGGGAAGGGCTTGAGATCAGACTTGGTATTTT
GTATGTTACTTCTTCGGGGGTGGCCTCTACAGTTTATCGGGCCAGCATCA
GTTTGGGCGGTAGGAGAATTGCGTTGGAATGTGGCACGGCTTCGGTTGTG
TGTTATAGCCTTCGTCGATACTGCCAGCCTAGACTGAGGACTGCGGTTTA
TACCTAGGATGTTGGCATAATGATCTTAAGTCGCCCGTCTTGAAACACGG
ACCAAGGAGTCTAACGTCTATGCGAGTGTTTGGGTGTAAAACCCGTACGC
GTAATGAAAGTGAACGTAGGTGGGGGCCCGTATGGGTGCACCATCGACC
GATCCTGATGTCTTCGGATGGATTTGAGTAAGAGCATAGCTGTTGGGACC
CGAAAGATGGTGAACTATGCCTGAATAGGGTGAAGCCAGAGGAAACTCT
GGTGGAGGCTCGTAGCGGTTCTGACGTGCAAATCGATCGTCGAATTTGGG
TATAGGGGCGAAAGACTAATCGACCC.

A nucleotide sequence of the Candida parapsilosis DNA fragments is shown as SEQ ID NO.27, and is specifically as follows:

GCATATCAATAAGCGGAGGAAAAGAAACCAACAGGGATTGCCTTA
GTAGCGGCGAGTGAAGCGGCAAAAGCTCAAATTTGAAATCTGGCACTTTC
AGTGTCCGAGTTGTAATTTGAAGAAGGTATCTTTGGGTCTGGCTCTTGTC
TATGTTTCTTGGAACAGAACGTCACAGAGGGTGAGAATCCCGTGCGATGA
GATGTCCCAGACCTATGTAAAGTTCCTTCGAAGAGTCGAGTTGTTTGGGA
ATGCAGCTCTAAGTGGGTGGTAAATTCCATCTAAAGCTAAATATTGGCGA
GAGACCGATAGCGAACAAGTACAGTGATGGAAAGATGAAAAGAACTTTG
AAAAGAGAGTGAAAAAGTACGTGAAATTGTTGAAAGGGAAGGGCTTGAGA
TCAGACTTGGTATTTTGTATGTTACTCTCTCGGGGGTGGCCTCTACAGTT
TACCGGGCCAGCATCAGTTTGAGCGGTAGGATAAGTGCAAAGAAATGTGG
CACTGCTTCGGTAGTGTGTTATAGTCTTTGTCGATACTGCCAGCTTAGAC
TGAGGACTGCGGCTTCGGCCTAGGATGTTGGCATAATGATCTTAAGTCGC
CCGTCTTGAAACACGGACC.

A nucleotide sequence of the Candida krusei DNA fragments is shown as SEQ ID NO.28, and is specifically as follows:

TTTCATGCGCTTGATTTTTCAAGCCAACCATCGAAAGGGAATCCGG
TTAAAATTCCGGAACTTGGATATGGATTCTTCACGGCAACGTAACTGAAT
GTGGAGACGTCGGCGTGAGCCCTGGGAGGAGTTATCTTTTCTTCTTAACA
GCTTATCACCCTGGAATTGGTTTATCCGGAGATGGGGTCTTATGGCTGGA
AGAGCGCGGTAATTTTGCCGCGTCCGGTGCGCTCACGACGGTCCTTGAAA
ATCCACAGGAAGGAATAGTTTTCATGCCAAGTCGTACTCATAACCGCAGC
AGGTCTCCAAGGTTAACAGCCTCTAGTTGATAGAATAATGTAGATAAGGG
AAGTCGGCAAAATAGATCCGTAACTTCGGGATAAGGATTGGCTCTAAGG
ATCGGGTGGTTTGGGCCTTGCGTAGAAGTGGTGGTGACTGGCGGCGGGCT
GCTTTCGGGCGGACTGCTGTTGGACGTCGCTATAGACACACTTGGTAGGC
ATTTATGTCGTCCGGATCACGCTTAACGATCAACTTAGAACTGGTACGGA
CAAGGGGAATCTGACTGTCTAATTAAAACATAGCATTGTGATGGTCAGAA
AGTGATGTTGACACAATGTGATTTCTGCCCAGTGCT.

A nucleotide sequence of the Candida glabrata DNA fragments is shown as SEQ ID NO.29, and is specifically as follows:

GCCTTAGTAACGGCGAGTGAAGCGGCAAAAGCTCAAATTTGAAAT
CTGGTACCTTTGGTGCCCGAGTTGTAATTTGGAGAGTACCACTTTGGGAC
TGTACTTTGCCTATGTTCCTTGGAACAGGACGTCATGGAGGGTGAGAATC
CCGTGTGGCGAGGTGTGTCAGTTCTTTGTAAAGGGTGCTCGAAGAGTCGA
GTTGTTTGGGAATGCAGCTCTAAGTGGGTGGTAAATTCCATCTAAAGCTA
AATACAGGCGAGAGACCGATAGCGAACAAGTACAGTGATGGAAAGATGA
AAAGAACTTTGAAAAGAGAGTGAAAAAGTACGTGAAATTGTTGAAAGGG
AAGGGCATTTGATCAGACATGGTGTTTTGCGCCTTGCCTCTCGTGGGGGG
ACTCTCGCAGCTCACTGGGCCAGCATCGGTTTTGGCGGCCGGAAAAAACC
TAGGGAATGTGGCTCTGCCTCGGTGTAGAGTTATAGCCCTGGGGAATACG
GCCAGCCGGGACCGAGGACTGCGATTTGTATCTAGGATGCTGGCATAATG
GTTATATGCCGCCCGTCTTGAAACACGGACCAAGGAGTCTAACGTCTATG
CGAGTGTTTGGGTGTTAAACCCGTACGCGTAATGAAAGTGAACGTAGGTT
GGGCCCCTGGGGGGTGCACAATCGACCGATCCTGATGTCTTCGGATGGAT
TTGAGTAAGAGCATAGCTGTTGGGACCCGAAAGATGGTGAACTATGCCTG
AATAGGGTGAAGCCAGAGGAAACTCTGGTGGAGGCTCGTAGCGGTTCTG
ACGTGCAAATCGATCGTCGAATTTGGGTATAGGGGCGAAAGACTAATCG
AACCATCTAGTAGCTGGTTCCTGCCGAAGTT.

Auxiliary reagents: a Taq enzyme, dNTP, MgCl₂, DMSO and a buffer solution.

Centrifuge tubes filled with the above components and instructions were packed in the kit.

Embodiment 2 Detection of Separate Candida Types

Separate Candida types were detected by the kit in Embodiment 1, and the detection method includes the following steps:

(1) sample DNA was extracted;

(2) a Taq enzyme, dNTP, MgCl₂, DMSO, a buffer solution, and specific primers, detection probes and complementary probes for five kinds of Candida were added into the sample DNA in the step (1); and preparing a system according to Table 2;

TABLE 2

| Component | Dosage | |
|---|---|---|
| TaqPolymerase | 2 | U |
| dNTP | 2M | |
| Primers (5 pairs) | 200 | nM*10 |
| Probes (5) | 100 | nM*5 |
| MgCl₂ | 3 | mM |
| Template | 1 | μL |
| DMSO | 5% | |
| Total | 26 | μL |

(3) the system in the step (2) was put into a real-time fluorescent PCR instrument; and a PCR amplification reaction was carried out, wherein specific conditions were as follows:

(1″) preheating was performed at 50° C. for 2 min, and 1 cycle was conducted;

(2″) pre-degeneration was performed at 95° C. for 10 min, and 1 cycle was conducted;

(3″) degeneration was performed at 95° C. for 10 s; extension was performed at 58° C. for 40 s; a fluorescence signal was acquired during extension; and 45 cycles were conducted; and

(4″) the temperature was maintained at 95° C. for 5 s and at 35° C. for 1 min; the temperature was raised to 97° C. at 0.2° C. per second; the fluorescence signal was continuously acquired for 5 times per ° C.

A fluorescence detection channel in the step (4″) was FAM; PCR procedures were operated; and a file was saved;

(4) Result determination:

1) Determination of Kit Effectiveness:

Negative quality control group effectiveness: the negative quality control group had no typical amplification curve or no Ct value in the channel FAM, otherwise reagent pollution may exist; determination should be performed after a pollution source was removed;

Positive quality control group effectiveness: all the positive quality control groups had typical amplification curves in the channel FAM; Ct values of the positive quality control were regulated to 20 by regulating thresholds after confirmation; and the Ct values were taken as thresholds of the to-be-detected samples;

2) Effectiveness Determination of Detected Samples:

If the Ct value existed, it was indicated that the added DNA had a normal amount;

3) Determination of Separate Candida Types:

After the products were subjected to cyclic PCR amplification, DNA sequences of different Candida types had different melting temperatures; instrument calibration was performed by virtue of five plasmids in the positive quality control; and the Candida types were detected by Roche 480 in the present invention so as to obtain a product annealing temperature table (Table 3) corresponding to Candida albicans, Candida tropicalis, Candida parapsilosis, Candida krusei and Candida glabrata. It should be indicated that, any instrument meeting the real-time fluorescent PCR was available. A specific limitation was not needed herein. A purpose of calibration was to eliminate specific annealing temperature differences brought by different instruments.

TABLE 3
Comparison table of annealing temperature
Tm of different Candida types

| Candida types | Melting temperature (Tm value) ° C. |
|---|---|
| Candida albicans | 63 |
| Candida tropicalis | 40 |
| Candida parapsilosis | 52 |
| Candida krusei | 46 |
| Candida glabrata | 58 |

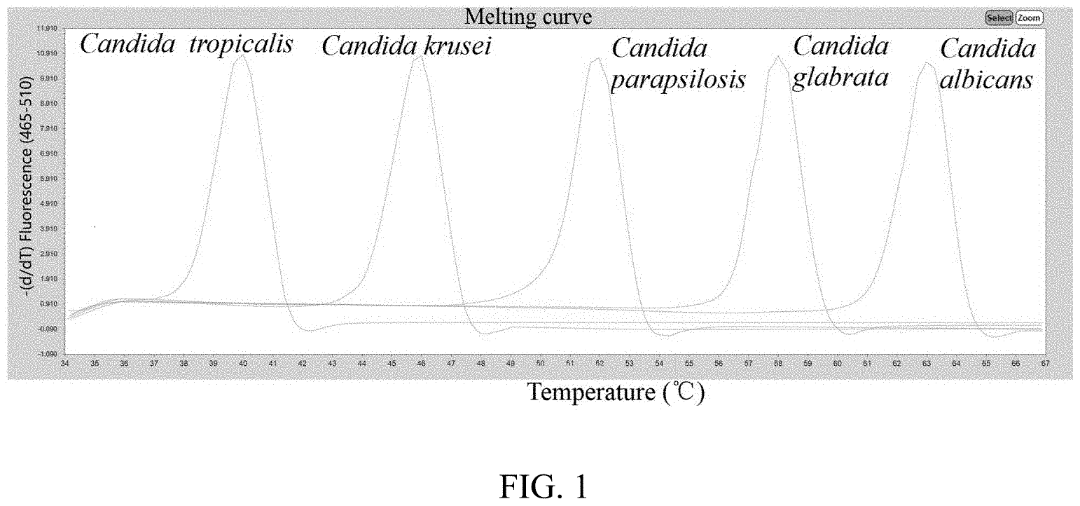

It can be seen from Table 2 that, the Tm values of the products of different Candida types have greater differences, which are up to 4° C. or higher. Thus, the Candida types are conveniently distinguished; the specificity is excellent; the sensitivity is high; and specific measured values that are not limited to the PCR instrument may have slight deviation, but the Tm values among the Candida types are relatively stable. By detecting the positive quality control, the Tm value comparison table applicable to any machine may be obtained; and a melting curve chart corresponding to the positive quality control is shown as FIG. 1.

Embodiment 3 Specificity Detection and Analysis

DNA of Cryptococcus neoformans, Cryptococcus gattii, Aspergillus fumigates, Aspergillus flavus, Aspergillus terreus, Aspergillus niger, Aspergillus nidulans, Aspergillus versicolor, Saccharomyces cerevisiae, Penicillium marneffei, Candida lusitaniae, Candida guilliermondii, Bordetella pertussis, Haemophilus influenza, Pseudomonas aeruginosa, Staphylococcus aureus and Streptococcus pneumonia was respectively extracted, and then detected by the kit in Embodiment 1 of the present invention according to the method in Embodiment 2. Results were shown in Table 4 below:

TABLE 4

| Name of strains | Detected Ct value | Detected Tm value |
|---|---|---|
| Cryptococcus neoformans | — | — |
| Cryptococcus gattii | — | — |
| Aspergillus fumigatus | — | — |
| Aspergillus flavus | — | — |
| Aspergillus terreus | — | — |
| Aspergillus niger | — | — |
| Aspergillus nidulans | — | — |
| Aspergillus versicolor | — | — |
| Saccharomyces cerevisiae | — | — |
| Penicillium marneffei | — | — |
| Candida lusitaniae | — | — |
| Candida guilliermondii | — | — |
| Bordetella pertussis | — | — |
| Haemophilus influenzae | — | — |
| Pseudomonas aeruginosa | — | — |
| Staphylococcus aureus | — | — |
| Streptococcus pneumoniae | — | — |

Cryptococcus, Aspergillus and Candida are clinically common fungi causing invasive fungal diseases. The above bacteria are common bacteria seeds causing bacterial infections in respiratory tracts and the like. It can be seen from Table 4 that, in presence of nucleic acids of the above fungi or bacteria, any amplification reaction is not carried out in the detection system in the present invention, and any specific melting curve or Tm value is not detected, which indicates that the present invention has excellent specificity.

Embodiment 4 Sensitivity Detection and Analysis

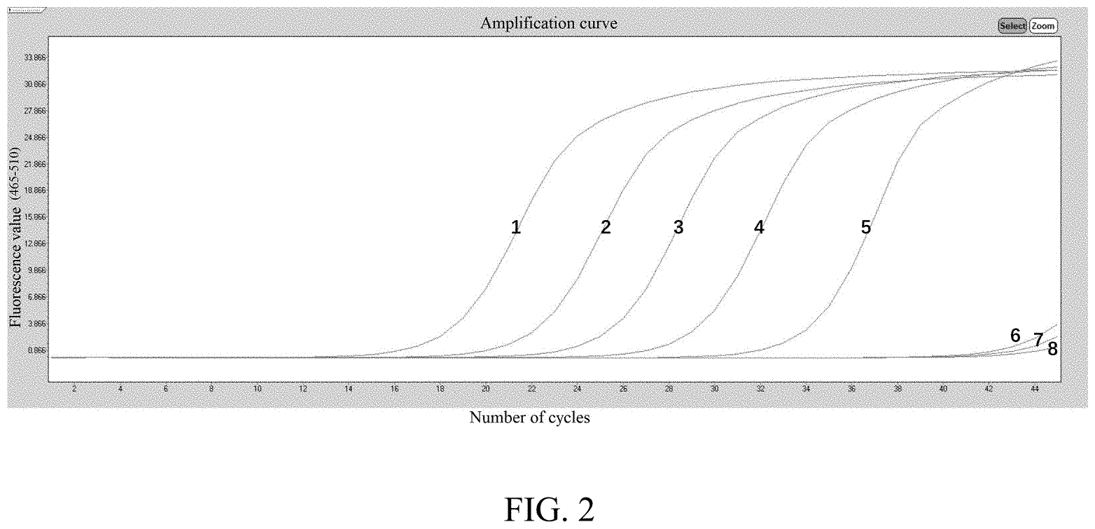

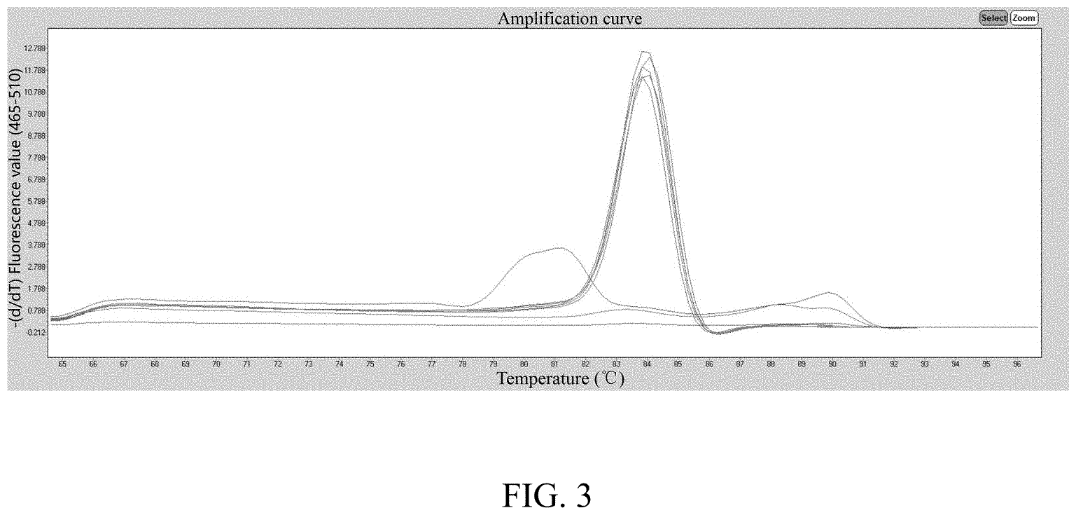

The sensitivity of plasmid standard substances of target sequences of five kinds of Candida of different concentrations was detected by a melting curve method; the plasmid standard substances (Shanghai Sangon Synthesis) of target sequences of synthetic Candida albicans, Candida tropicalis, Candida parapsilosis, Candida krusei and Candida glabrata were diluted into 10⁶ copies, 10⁵ copies, 10⁴ copies, 10³ copies, 10² copies, 10¹ copies and 10⁰ copies. The plasmid standard substances were taken as templates, detected by the kit in Embodiment 1 and the method in Embodiment 2 by utilizing a Roche 48011 fluorescent quantitative PCR instrument, and then subjected to melting curve analysis. By taking the Candida albicans as an example, amplification curves of different concentrations were shown as FIG. 2, wherein curves 1-8 respectively corresponded to 10⁶ copies, 10⁵ copies, 10⁴ copies, 10³ copies, 10² copies, 10¹ copies, 10⁰ copies and negative control; and the melting curve was shown as FIG. 3.

It can be seen from the amplification curve in FIG. 2, in presence of the plasmid standard substances of 10⁶ copies-10² copies, an excellent signal can be given in the present invention; in presence of the plasmid standard substances of 10¹ copies-10⁰ copies, a fluorescence signal cannot be detected; and in absence of the target sequence, the fluorescence signal cannot be detected in the present invention. Through the melting curve in FIG. 3, it is indicated that, in presence of the plasmid standard substances of 10⁶ copies-10² copies, unique and consistent melting points can be formed, and a single peak in the figure is formed; in presence of the plasmid standard substances of 10¹ copies-10⁰ copies, a unique and consistent single peak cannot be formed; and in absence of the target sequence, there is no single peak. Therefore, the sensitivity of the plasmid standard substances for detecting the target sequences of the Candida by the melting curve method in the present invention may be up to 10² copies.

Embodiment 5 Quantitative Analysis

Five kinds of Candida were quantitatively detected, such as Candida albicans, Candida tropicalis, Candida parapsilosis, Candida krusei and Candida glabrata.

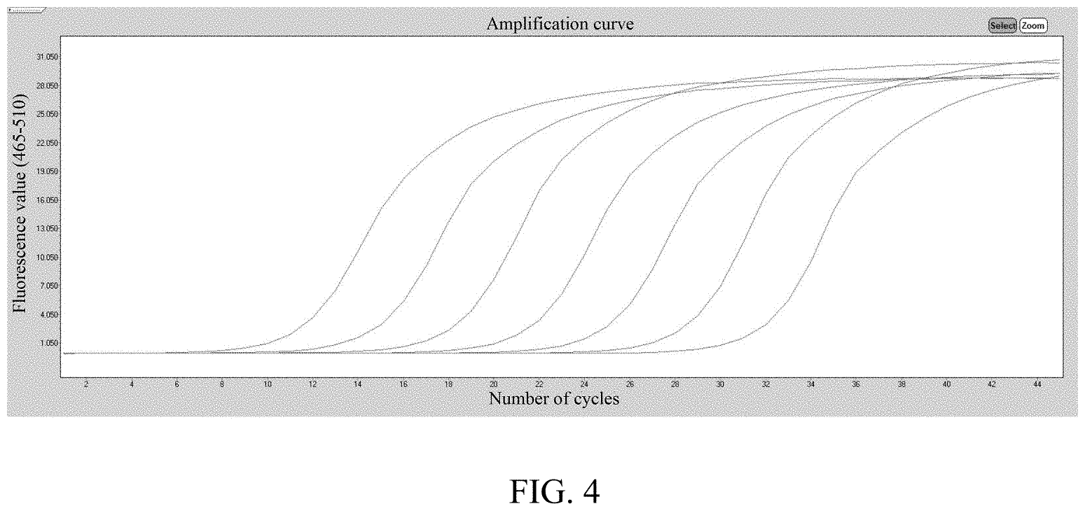

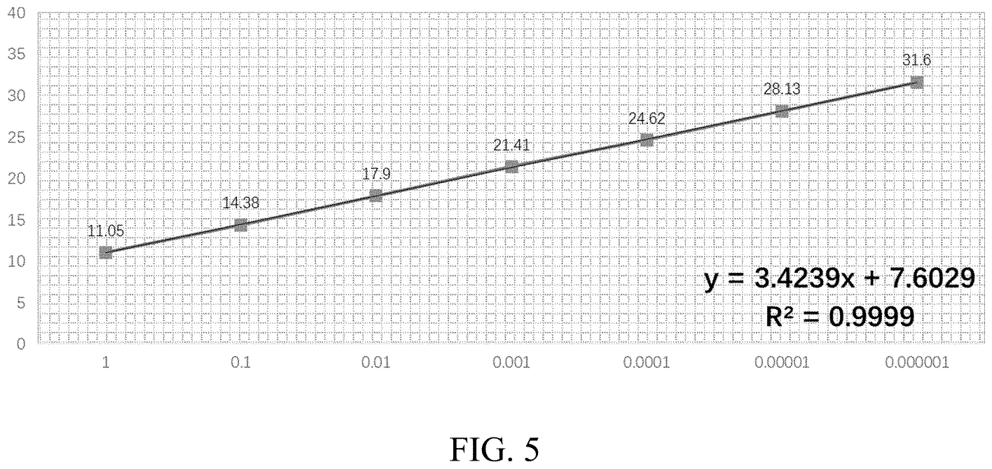

Each of the five kinds of Candida that can be detected by the kit was equivalently and uniformly mixed at a respective concentration of 1 ng/μl, and subjected to 10-times continuous gradient dilution so as to prepare a standard curve of 7 concentration points; each concentration was subjected to 3 duplications; arabidopsis gene fragments were taken as negative control; an amplification curve was shown as FIG. 4 (taking Candida albicans as an example); and a linear relation between the Ct value and the concentration was shown as FIG. 5.

It can be seen from FIG. 5 that, a standard curve formed a linear equation y=3.4329x+7.6029 in a linear amplification region of the PCR reaction; R2 was equal to 0.9999; amplification efficiency of the kit was 95.9%; and a linear range was 1 ng/μl-0.000001 ng/μl. When the samples were measured, the above standard curve, the negative control and sample DNA of an unknown concentration were subjected to fluorescent PCR reactions; and when the fluorescent quantitative PCR instruments such as Roche LightCycler 48011, ABI7500 and StepOne Plus were used, a nucleic acid concentration of target genes in the samples can be calculated by built-in analysis software according to the standard curve and the Ct value of the sample, thereby conducting quantitative analysis.

Embodiment 6 Stability Detection

1) A stability detection kit-Candida albicans was formed by utilizing the auxiliary reagents in Embodiment 1, and specific primers, a detection probe and a complementary probe for the Candida albicans according to components and dosages in the following table; a stability detection kit-Candida parapsilosis was formed by utilizing the auxiliary reagents in Embodiment 1, and specific primers, a detection probe and a complementary probe for the Candida parapsilosis according to components and dosages in the following table; a stability detection kit-Candida tropicalis was formed by utilizing the auxiliary reagents in Embodiment 1, and specific primers, a detection probe and a complementary probe for the Candida tropicalis according to components and dosages in the following table; a stability detection kit-Candida krusei was formed by utilizing the auxiliary reagents in Embodiment 1, and specific primers, a detection probe and a complementary probe for the Candida krusei according to components and dosages in the following table; and a stability detection kit-Candida glabrata was formed by utilizing the auxiliary reagents in Embodiment 1, and specific primers, a detection probe and a complementary probe for the Candida glabrata according to components and dosages in the following table. A separate detection system for each kind of Candida was shown as Table 5:

TABLE 5

| Component | Dosage | |
|---|---|---|
| TaqPolymerase | 2 | U |
| dNTP | 2M | |
| Primers (1 pair) | 200 | nM*2 |
| Probe (1) | 100 | nM |
| MgCl₂ | 3 | mM |
| Template | 1 | μL |
| DMSO | 5% | |
| Total | 25 | μL |

2) DNA of the cultured Candida albicans, Candida tropicalis, Candida parapsilosis, Candida krusei and Candida glabrata was extracted;

3) Ct values of the DNA of the above strains detected by the kit in Embodiment 1 were respectively compared with Ct values of corresponding strains in 2) detected by the five stability detection kits in 1) by utilizing the method in Embodiment 2, i.e., results of the detected Ct values of the DNA of the five kinds of Candida in Embodiment 1 and the detected Ct values of the DNA of the five kinds of Candida by the stability detection kits were compared; the standard deviation and CV values of the two groups of results were analyzed and compared; and the results were shown as Table 6 as follows:

TABLE 6

| | Candida | Candida | Candida | Candida | Candida |
|---|---|---|---|---|---|
| | albicans | parapsilosis | tropicalis | krusei | glabrata |
| Detection | 23.12 ± | 25.56 ± | 21.26 ± | 20.76 ± | 19.98 ± |
| kit | 0.05 | 0.06 | 0.04 | 0.05 | 0.06 |
| Stability | 23.13 ± | 25.55 ± | 21.25 ± | 20.74 ± | 19.97 ± |
| kit | 0.03 | 0.08 | 0.05 | 0.04 | 0.08 |
| STD | 0.05 | 0.06 | 0.04 | 0.05 | 0.06 |
| CV % | <3 | <3 | <3 | <3 | <3 |

It can be seen from Table 6 that, the complex primer and probe system designed in the detection kit has the detection efficiency consistent with that of a separate system; and the detection performance in the complex system is stable.

The applicant declares that, the detailed method of the present invention is described through the above embodiments in the present invention. However, the present invention is not limited to the above detailed method, which means that the present invention can be implemented without depending on the above detailed method. It can be understood by those skilled in the art that, any improvements of the present invention, equivalent replacements of various raw materials of the product in the present invention, addition of auxiliary components and selection of specific manners should be included in the protection scope and disclosure scope of the present invention.

1. A primer probe combination for detecting separate Candida types, comprising specific primers, detection probes and complementary probes specified at Candida albicans, Candida tropicalis, Candida parapsilosis, Candida krusei and Candida glabrata respectively, wherein the complementary probes and the detection probes are in incomplete complementary pairing;
nucleotide sequences of the specific primers of the Candida albicans are shown as SEQ ID NO.1-2; nucleotide sequences of the specific primers of the Candida tropicalis are shown as SEQ ID NO.3-4; nucleotide sequences of the specific primers of the Candida parapsilosis are shown as SEQ ID NO.5-6; nucleotide sequences of the specific primers of the Candida krusei are shown as SEQ ID NO.7-8; and nucleotide sequences of the specific primers of the Candida glabrata are shown as SEQ ID NO.9-10;
a nucleotide sequence of the detection probe of the Candida albicans is shown as SEQ ID NO.11; a nucleotide sequence of the detection probe of the Candida tropicalis is shown as SEQ ID NO.12; a nucleotide sequence of the detection probe of the Candida parapsilosis is shown as SEQ ID NO.13; a nucleotide sequence of the detection probe of the Candida krusei is shown as SEQ ID NO.14; and a nucleotide sequence of the detection probe of the Candida glabrata is shown as SEQ ID NO.15;
a nucleotide sequence of the complementary probe of the Candida albicans is shown as SEQ ID NO.16; a nucleotide sequence of the complementary probe of the Candida tropicalis is shown as SEQ ID NO.17; a nucleotide sequence of the complementary probe of the Candida parapsilosis is shown as SEQ ID NO.18; a nucleotide sequence of the complementary probe of the Candida krusei is shown as SEQ ID NO.19; and a nucleotide sequence of the complementary probe of the Candida glabrata is shown as SEQ ID NO.20.

2. The primer probe combination according to claim 1, wherein the primer probe combination further comprises an internal reference system; the internal reference system comprises internal reference primers and an internal reference probe; nucleotide sequences of the internal reference primers are shown as SEQ ID NO.21-22; and a nucleotide sequence of the internal reference probe is shown as SEQ ID NO.23.

3. The primer probe combination according to claim 1, wherein a quencher is modified at 3′ end of the detection probe of the Candida albicans, Candida tropicalis, Candida parapsilosis, Candida krusei and Candida glabrata; and a fluorophore is modified at 5′ end.

4. The primer probe combination according to claim 3, wherein the fluorophore comprises any one or a combination of at least two of ALEX-350, Alexa Fluor 488, CY3, FAM, VIC, TET, CALGold540, JOE, HEX, CALFluorOrange560, TAMRA, CALFluorRed590, ROX, CALFluorRed610, TexasRed, CALFluorRed635, Quasar670, CY5, CY5.5, LC RED640 or Quasar705; and the quencher modified at the 3′ end of the detection probe comprises any one or a combination of at least two of TAMRA, DABCYL, BHQ-1, BHQ-2, BHQ-3 or Eclipse.

5. The primer probe combination according to claim 1, wherein the 3′ end of the complementary probe is modified with the quencher; and the quencher modified at the 3′ end of the complementary probe comprises any one or a combination of at least two of TAMRA, DABCYL, BHQ-1, BHQ-2, BHQ-3 or Eclipse.

6. A kit for detecting separate Candida types, comprising the primer probe combination of claim 1, and further comprising negative quality control, positive quality control and auxiliary reagents, wherein the negative quality control is plasmids containing arabidopsis DNA fragments; a nucleotide sequence of the arabidopsis DNA fragments is shown as SEQ ID NO.24; the positive quality control is respectively a plasmid containing Candida albicans DNA fragments, a plasmid containing Candida tropicalis DNA fragments, a plasmid containing Candida parapsilosis DNA fragments, a plasmid containing Candida krusei DNA fragments, and a plasmid containing Candida glabrata DNA fragments;
nucleotide sequences of the Candida albicans DNA fragments, the Candida tropicalis DNA fragments, the Candida parapsilosis DNA fragments, the Candida krusei DNA fragments and the Candida glabrata DNA fragments are respectively shown as SEQ ID NO.25, SEQ ID NO.26, SEQ ID NO.27, SEQ ID NO.28 and SEQ ID NO.29; the auxiliary reagents comprise a Taq enzyme, dNTP, MgCl₂, DMSO and a buffer solution; the buffer solution comprises Tris-HCl and KCl; a concentration of the Tris-HCl in the buffer solution is 15-20 mM; and a concentration of the KCl in the buffer solution is 100-200 mM.

7. A method for detecting separate Candida types by utilizing the kit of claim 6 for a purpose of non-disease diagnosis, comprising the following steps:
(1) extracting sample DNA;
(2) adding a Taq enzyme, dNTP, MgCl₂, DMSO, a buffer solution and the primer probe combination of five kinds of Candida in claim 1 into the sample DNA in the step (1); and
(3) carrying out a real-time fluorescent PCR reaction.

8. The method according to claim 7, wherein in the step (2), a final concentration of the DNA sample is 0.1 pg-10 ng; a final concentration of the Taq enzyme is 0.5-5 U; a final concentration of the dNTP is 0.1-5 M; a volume percentage of the DMSO is 1-8%; a volume percentage of the DMSO is 5%; a final concentration of specific primers of the five kinds of Candida is 50-500 nM; a final concentration of a detection probe of the five kinds of Candida is 50-500 nM; and a final concentration of a complementary probe of the five kinds of Candida is 50-500 nM.

9. The method according to claim 7, wherein conditions of the real-time fluorescent PCR reaction in the step (3) are as follows:
(1′) pre-degeneration at 90-98° C. for 1-10 min, and 1 cycle;
(2′) degeneration at 90-98° C. for 10 s; extension at 55-60° C. for 35-45 s; and 40-50 cycles; and
(3′) heating at 35-97° C.; and 1 cycle;
before the step (1′), the reaction further comprises a preheating process: 50° C. for 2 min; and
1 cycle.

10. The method according to claim 7, wherein the real-time fluorescent PCR reaction in the step (3) specifically comprises the following steps:
(1″) preheating at 50° C. for 2 min, and conducting 1 cycle;
(2″) performing pre-degeneration at 95° C. for 10 min, and conducting 1 cycle;
(3″) performing degeneration at 95° C. for 10 s; performing extension at 58° C. for 40 s; acquiring a fluorescence signal during extension; and conducting 45 cycles;
(4″) maintaining the temperature at 95° C. for 5 s and at 35° C. for 1 min; raising the temperature to 97° C. at 0.2° C. per second; continuously acquiring the fluorescence signal for 5 times per ° C.

11. The method according to claim 7, wherein after the step (3), the method further comprises a judgment step; and judgment standards are as follows:
(a) if the Ct value of the detection channel is not greater than 35, the result is positive; and if the Ct value of the detection channel is greater than 35, the result is negative; and
(b) the positive result is further analyzed; by virtue of melting curve analysis, corresponding annealing temperature values Tm of the product and five kinds of positive quality control are contrasted; and if the Tm values are the same, to-be-detected samples are judged to contain Candida types corresponding to the positive quality control.

12. An application of the primer probe combination of claim 1 in detection of separate Candida types or preparation of a reagent for detection of separate Candida types for a purpose of non-disease diagnosis.


SEQUENCE LISTING (29 sequences)

SEQ ID NO: 1 - DNA, 18 bp, Artificial Sequence
  Candida albicans forward primer, fully synthetic
tgtttgagcgtcgtttct

SEQ ID NO: 2 - DNA, 18 bp, Artificial Sequence
  Candida albicans reverse primer, fully synthetic
aagttcagcgggtagtcc

SEQ ID NO: 3 - DNA, 20 bp, Artificial Sequence
  Candida tropicalis forward primer, fully synthetic
gcctgtttgagcgtcgtttc

SEQ ID NO: 4 - DNA, 20 bp, Artificial Sequence
  Candida tropicalis reverse primer, fully synthetic
cgccttaccactaccgtctt

SEQ ID NO: 5 - DNA, 19 bp, Artificial Sequence
  Candida parapsilosis forward primer, fully synthetic
ctgtttgagcgtcgtttct

SEQ ID NO: 6 - DNA, 19 bp, Artificial Sequence
  Candida parapsilosis reverse primer, fully synthetic
taagttcagcgggtagtcc

SEQ ID NO: 7 - DNA, 20 bp, Artificial Sequence
  Candida krusei forward primer, fully synthetic
agtactacactgcgtgagcg

SEQ ID NO: 8 - DNA, 20 bp, Artificial Sequence
  Candida krusei reverse primer, fully synthetic
tgcgagaaccaagagatccg

SEQ ID NO: 9 - DNA, 20 bp, Artificial Sequence
  Candida glabrata forward primer, fully synthetic
aacgcagcgaaatgcgatac

SEQ ID NO: 10 - DNA, 20 bp, Artificial Sequence
  Candida glabrata reverse primer, fully synthetic
ggaataccagagggcgcaat

SEQ ID NO: 11 - DNA, 25 bp, Artificial Sequence
  Candida albicans detection probe, fully synthetic
atcactgtacttgttcgctatcggt

SEQ ID NO: 12 - DNA, 25 bp, Artificial Sequence
  Candida tropicalis detection probe, fully synthetic
tttccactcattggtacaaactcca

SEQ ID NO: 13 - DNA, 20 bp, Artificial Sequence
  Candida parapsilosis detection probe, fully synthetic
acgcacactcccaggtcttt

SEQ ID NO: 14 - DNA, 27 bp, Artificial Sequence
  Candida krusei detection probe, fully synthetic
tcctccgtctatgctccgttacctgtt

SEQ ID NO: 15 - DNA, 24 bp, Artificial Sequence
  Candida glabrata detection probe, fully synthetic
aaacgcttattttgctagtggcca

SEQ ID NO: 16 - DNA, 25 bp, Artificial Sequence
  Candida albicans complementary probe, fully synthetic
tagtgacatgaacaagcgataacca

SEQ ID NO: 17 - DNA, 25 bp, Artificial Sequence
  Candida tropicalis complementary probe, fully synthetic
aaaggcgagaaaccaagttagaggt

SEQ ID NO: 18 - DNA, 20 bp, Artificial Sequence
  Candida parapsilosis complementary probe, fully synthetic
tgcgagtgagggaccagaaa

SEQ ID NO: 19 - DNA, 27 bp, Artificial Sequence
  Candida krusei complementary probe, fully synthetic
aggatgcacatacgagacaatggacaa

SEQ ID NO: 20 - DNA, 24 bp, Artificial Sequence
  Candida glabrata complementary probe, fully synthetic
tttgcgaataaaaccatcaccggt

SEQ ID NO: 21 - DNA, 18 bp, Artificial Sequence
  Forward primer of internal reference primer, fully synthetic
ctgtgaatgccatttctc

SEQ ID NO: 22 - DNA, 18 bp, Artificial Sequence
  Reverse primer of internal reference primer, fully synthetic
acacctcctttggaatag

SEQ ID NO: 23 - DNA, 24 bp, Artificial Sequence
  Internal reference probe, fully synthetic
cctaacctacccgccttggatatt

SEQ ID NO: 24 - DNA, 809 bp, Artificial Sequence
  DNA fragment from arabidopsis, fully synthetic
catgattcagccaacataacccgacccgtacaattctattacgcgtctccggcgactgactatacttgccgttcggagttagggttttactccgagagaaaattgagtcagcgatgaatccgttgacaaggtgaagaagacgcagagcattaacgcgaaagaattagatctagggatttacgacgaagcatcttggcacgccaagtacaaagattcagcctacgtttacgtcggagaattaccttacgatctcacggagggtgacctcctcgccgttttctcacaatatggtgaagttgttgatttgaatcttgttcgagataaaggaactgggagatcaaaaagatttgcgtttgttgcttatgaagatcagagaagtactaatcttgctgttggataataaaagtggagcattgtggtaaatacttaaagagagaagaggaagatgaagagacgaagcagaagaagagagaagctccgtggtgtttgcagagcttttcagagaaaggagtgtactcgtggagattcttgcaaattttctcacgatgaaaatagagctgccaataccgggtggggtcacgaagatcgtagaagttccaagtgagagatcaagtaagtaaataccataatgatgtagaggataaatagggattgttcatattgatatccaagtggtaatgctctacattgaagaatggtttctaagttgtagctagaatctaatggaaaatgtgatttattgaccattagcttacacaccggtgttttgctctgtatatgtcgatcttattttcagtgttcacctttac

SEQ ID NO: 25 - DNA, 593 bp, Artificial Sequence
  DNA fragment from Candida albicans, fully synthetic
atatcaataagcggaggaaaagaaaccaacagggattgcctcagtagcggcgagtgaagcggcaaaagctcaaatttgaaatctggcgtctttggcgtccgagttgtaatttgaagaaggtatctttgggcccggctcttgtctatgttccttggaacaggacgtcacagagggtgagaatcccgtgcgatgagatgacccgggtctgtgtaaagttccttcgacgagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcttgagatcagacttggtattttgcatgctgctctctcgggggcggccgctgcggtttaccgggccagcatcggtttggagcggcaggataatggcggaggaatgtggcacggcttctgctgtgtgttatagcctctgacgatactgccagcctagaccgaggactgcggtttttacctaggatgttggcataatgatcttaagtcgc

SEQ ID NO: 26 - DNA, 865 bp, Artificial Sequence
  DNA fragment from Candida tropicalis, fully synthetic
aaaagaaaccaacagggattgccttagtagcggcgagtgaagcggcaaaagctcaaatttgaaatctggctctttcagagtccgagttgtaatttgaagaaggtatctttgggtctggctcttgtctatgtttcttggaacagaacgtcacagagggtgagaatcccgtgcgatgagatgatccaggcctatgtaaagttccttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcttgagatcagacttggtattttgtatgttacttcttcgggggtggcctctacagtttatcgggccagcatcagtttgggcggtaggagaattgcgttggaatgtggcacggcttcggttgtgtgttatagccttcgtcgatactgccagcctagactgaggactgcggtttatacctaggatgttggcataatgatcttaagtcgcccgtcttgaaacacggaccaaggagtctaacgtctatgcgagtgtttgggtgtaaaacccgtacgcgtaatgaaagtgaacgtaggtgggggcccgtatgggtgcaccatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaccc

SEQ ID NO: 27 - DNA, 613 bp, Artificial Sequence
  DNA fragment from Candida parapsilosis, fully synthetic
gcatatcaataagcggaggaaaagaaaccaacagggattgccttagtagcggcgagtgaagcggcaaaagctcaaatttgaaatctggcactttcagtgtccgagttgtaatttgaagaaggtatctttgggtctggctcttgtctatgtttcttggaacagaacgtcacagagggtgagaatcccgtgcgatgagatgtcccagacctatgtaaagttccttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcttgagatcagacttggtattttgtatgttactctctcgggggtggcctctacagtttaccgggccagcatcagtttgagcggtaggataagtgcaaagaaatgtggcactgcttcggtagtgtgttatagtctttgtcgatactgccagcttagactgaggactgcggcttcggcctaggatgttggcataatgatcttaagtcgcccgtcttgaaacacggacc

SEQ ID NO: 28 - DNA, 631 bp, Artificial Sequence
  DNA fragment from Candida krusei, fully synthetic
tttcatgcgcttgatttttcaagccaaccatcgaaagggaatccggttaaaattccggaacttggatatggattcttcacggcaacgtaactgaatgtggagacgtcggcgtgagccctgggaggagttatcttttcttcttaacagcttatcaccctggaattggtttatccggagatggggtcttatggctggaagagcgcggtaattttgccgcgtccggtgcgctcacgacggtccttgaaaatccacaggaaggaatagttttcatgccaagtcgtactcataaccgcagcaggtctccaaggttaacagcctctagttgatagaataatgtagataagggaagtcggcaaaatagatccgtaacttcgggataaggattggctctaaggatcgggtggtttgggccttgcgtagaagtggtggtgactggcggcgggctgctttcgggcggactgctgttggacgtcgctatagacacacttggtaggcatttatgtcgtccggatcacgcttaacgatcaacttagaactggtacggacaaggggaatctgactgtctaattaaaacatagcattgtgatggtcagaaagtgatgttgacacaatgtgatttctgcccagtgct

SEQ ID NO: 29 - DNA, 872 bp, Artificial Sequence
  DNA fragment from Candida glabrata, fully synthetic
gccttagtaacggcgagtgaagcggcaaaagctcaaatttgaaatctggtacctttggtgcccgagttgtaatttggagagtaccactttgggactgtactttgcctatgttccttggaacaggacgtcatggagggtgagaatcccgtgtggcgaggtgtgtcagttctttgtaaagggtgctcgaagagtcgagttgtttgggaatgcagctctaagtgggtggtaaattccatctaaagctaaatacaggcgagagaccgatagcgaacaagtacagtgatggaaagatgaaaagaactttgaaaagagagtgaaaaagtacgtgaaattgttgaaagggaagggcatttgatcagacatggtgttttgcgccttgcctctcgtggggggactctcgcagctcactgggccagcatcggttttggcggccggaaaaaacctagggaatgtggctctgcctcggtgtagagttatagccctggggaatacggccagccgggaccgaggactgcgatttgtatctaggatgctggcataatggttatatgccgcccgtcttgaaacacggaccaaggagtctaacgtctatgcgagtgtttgggtgttaaacccgtacgcgtaatgaaagtgaacgtaggttgggcccctggggggtgcacaatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtt